(19) United States
(12) Patent Application Publication
(10) Pub. No.: US 2026/0053706 A1
(43) Pub. Date: Feb. 26, 2026

(54) LOCK TO POLE BIOMETRIC ACCESS INTRAVENEOUS MEDICATION LOCKBOX

(71) Applicant: Health Research, Inc., Buffalo, NY (US)

(72) Inventors: Andrew STORER, Clarence, NY (US); Jillianna WASIURA, West Seneca, NY (US)

(21) Appl. No.: 19/102,821

(22) PCT Filed: Aug. 21, 2023
(86) PCT No.: PCT/US2023/072600

§ 371 Date: Feb. 10, 2025

Related U.S. Application Data
(60) Provisional application No. 63/371,929, filed on Aug. 19, 2022.

(51) Int. Cl. A61J 1/16; A61J 1/14; G16H 20/13; G16H 40/20; G16H 40/40

(52) U.S. Cl. CPC A61J 1/16; A61J 1/1412; A61J 1/1437; G16H 20/13; G16H 40/20; G16H 40/40

(57) ABSTRACT

A container is provided for securing IV medication during use. The container includes a housing with a removable cover. The housing has an internal volume for placement of IV medication. In other words, the housing and the cover form an internal volume when the container is closed. The container has a biometric lock for selectively locking the cover to the housing. The biometric lock may be a fingerprint lock. The biometric lock may be configured to store an access log. In another aspect, a method for securing IV medication during use is provided.

### CROSS-REFERENCE TO RELATED APPLICATIONS

This application claims priority to U.S. Provisional Application No. 63/371,929, filed on Aug. 19, 2022, now pending, the disclosure of which is incorporated herein by reference.

### FIELD OF THE DISCLOSURE

The present disclosure relates to medication management, and in particular, to techniques for securing IV medications during use.

### BACKGROUND OF THE DISCLOSURE

The opioid crisis has reached unprecedented levels and hospitals have a responsibility to ensure accurate tracking, safeguarding, use, and access to medications. Per federal law (The Controlled Substance Act and the Code of Federal Regulations) accrediting bodies (DNV, Joint Commission), and professional organization recommendations/guidelines (Infusion Nursing Society) institutions that utilize controlled substances must have a diversion prevention program. To combat the increasing diversion problem (substance use disorder (SUD) has been described as affecting 10-25% of healthcare workers) federal agencies have stressed the importance of drug diversion programs by fining healthcare institutions unprecedented levels (Federal DEA Fine is $10,000 or more for each violation including failure to keep accurate record. Recent examples include University of Michigan: $4.3 million and Massachusetts General Hospital $2.3 million). The first role of a diversion prevention program is primary prevention. Primary prevention aims to prevent injury before it occurs. Therefore, one of the main aims of a diversion prevention program is to ensure that employees have the proper resources to prevent diversion and to secure medications access so that only those who need access at the appropriate time are allowed access. Access to medications is considered one of the chief causative factors in drug diversion. An additional role that diversion prevention programs has is to track medication access and identify gaps or discrepancies in drug disposition (administration, waste, or return).

Relevant points of the U.S. federal regulation include:
Drugs and biologicals must not be stored in areas that are readily accessible to unauthorized persons
If there is evidence of tampering or diversion, or if medication security otherwise becomes a problem, the hospital is expected to evaluate its current medication control policies and procedures, and implement the necessary systems and processes to ensure that the problem is corrected, and that patient health and safety are maintained
All controlled substances must be locked and accessible by authorized personnel.

Regulations and guidance can be found at, for example, 42 CFR § 482.25(b) (Standard: Delivery of Services), 42 CFR § 482.25(b)(2)(i) (All drugs and biologicals must be kept in a secure area, and locked when appropriate), and Infusion Therapy Standards of Practice 5.1 and 5.2 (published by the Infusion Nurses Society (INS)).

Prior devices for securing narcotic and other infusions at the bedside include a translucent IV pole locked medication box opened via a key or manual code. However, the use of keys is problematic because master/universal keys are simple and commonly available, often shared between personnel such that the actual user is unknown, and easily misplaced. The current practice in most institutions is to secure the key in the automated medication dispensing cabinet. The medical/nursing staff then “pull” the key under their login into the medication dispensing system. Given the timeliness of medications “running out” or inability to return the key many staff “share” the key. In addition, a quick Google Search and those that may want access to the lockbox can find “universal” keys easily for sale on the internet. The general process adds additional steps and significant time (for a patient with 1 infusion that requires changing every four hours, a nurse working a 12-hour shift would spend approximately 10-15 per day locating, accessing, and returning the key) into the workday that could be geared towards providing patient care. The staff member has to go to medication room, login, grab key (and if not present ask around to who has), obtain medication from automated dispensing system or other source, go unlock box, hang medication, program pump and then return the key. Many hospitalized patients are also on “isolation precautions” designed at protecting them from infection if they are immunocompromised or aimed at protecting the staff from easily transmittable infections (COVID-19, C-Diff, Monkeypox).

The use of codes (e.g., 4-digit numeric passcodes) is problematic because the codes are often universal (same code is used across devices in a hospital unit) and/or shared between personnel, can be viewed and stolen by unauthorized staff, patients, or family members.

There is a need for tools to improve the ability to secure and trace medications including at the time of patient use.

### BRIEF SUMMARY OF THE DISCLOSURE

In a first aspect, the present disclosure provides a container for securing IV medication during use. The container includes a housing with a removable cover. The cover may be hingedly attached to the housing. The cover may be transparent (e.g., translucent), tinted, or both. The housing has an internal volume for placement of IV medication. In other words, the housing and the cover form an internal volume when the cover is attached to the housing (i.e., when the container is closed). The container has a biometric lock for selectively locking the cover to the housing. The biometric lock may be a fingerprint lock. The biometric lock may be configured to store an access log. The container may include a hangar for holding an IV bag.

The container has a fastener for affixing the housing to an IV pole. The fastener may be configured to be removable and is only operable from within the housing. The fastener may be, for example, one or more of a clamp, a screw, a bolt, a latch, a buckle, and a strap, or any other suitable fastener or combination of fasteners. In some embodiments, the fastener permanently attaches the housing to an IV pole. In such embodiments, the IV pole may make up a part of the claimed device.

The container may further include a processor. A communication port may be in electronic communication with the processor. The processor may be configured to communicate with a medication management system by way of the communication port.

In some embodiments, the container includes an alarm. The alarm may be one or more of an audible alarm, a visual alarm, or other alarm type. The alarm may be configured to be triggered when the cover has been removed from the housing (i.e., the container is open) for a time greater than a predetermined period of time. The alarm may be configured to be further triggered when the cover has been removed from the housing for a time greater than a second predetermined period of time.

In some embodiments, the container includes a battery. The battery may be a rechargeable battery.

### DESCRIPTION OF THE DRAWINGS

For a fuller understanding of the nature and objects of the disclosure, reference should be made to the following detailed description taken in conjunction with the accompanying drawings.

FIG. 1 is a container according to an embodiment of the present disclosure.

FIG. 2 is a chart depicting a method according to the present disclosure.

### DETAILED DESCRIPTION OF THE DISCLOSURE

The present disclosure provides an electronic/smart lock-to-pole medication lockbox that includes features to prevent drug diversion through best practices for securing medications. Some embodiments of the device include the ability to track and limit the people having access to the device (including when and how often the lockbox was accessed), use of electricity to power the device with a battery backup, and use of biometric identification to open device, automatic locking system, and an alarm (for example, triggered if device is not secured properly within an appropriate time frame).

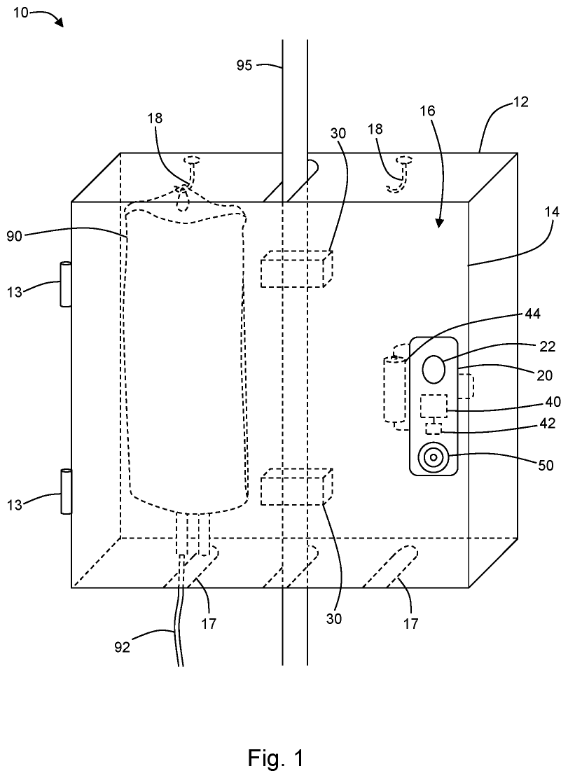

With reference to FIG. 1, the present disclosure may be embodied as a container 10 for securing intravenous (IV) medication 90 during use. The IV medication may be an IV bag (IV bag 90), a bottle, a syringe, etc. The container 10 has a housing 12 with a removable cover 14. The housing 12 and the cover 14 cooperate to form an internal volume 16 wherein IV medication can be placed during use (e.g., while being infused to a patient). As such, the housing 12 and/or the cover 14 may include one or more passages 17 through which an IV tube 92 may pass such that medication may flow from the IV bag 90 (or other container) to the patient. The passages 17 are configured such that an IV bag 90 cannot be removed through the passages (i.e., will not fit through a passage when the bag contains medication).

The cover 14 is removable such that IV medication may be inserted into or removed from the internal volume 16 of the container. For example, the cover 14 may be hingedly attached to the housing 12 (see hinges 13). Other techniques for removably attaching the cover to the housing may be used (e.g., pins, latches, tabs, hooks, etc.) The cover (or at least a portion of the cover) may be transparent. The cover (or at least a portion of the cover) may be tinted (for example, transparent and tinted to be amber, green, blue or other color). In this way, photosensitive medications can be protected from certain wavelengths of light. At least a portion of the container (e.g., at least a portion of the cover and/or at least a portion of the housing) is transparent. At least a portion of the container (e.g., at least a portion of the cover and/or at least a portion of the housing) is transparent. At least a portion of the container (e.g., at least a portion of the cover and/or at least a portion of the housing) is transparent and tinted.

The container 10 may include one or more hangars 18 for holding an IV bag 90. For example, the container may have one or more hooks from which one or more IV bags can be suspended. In another example, the container may include a rod from which one or more IV bags can be suspended. Other examples of hangars include, but are not limited to, straps, hook-and-look fasteners/straps, snaps, shelves, etc.

The container 10 includes a biometric lock 20 for selectively locking the cover 14 to the housing 12. In this way, the internal volume 16 cannot be accessed by a person other than by unlocking the lock 20. In some embodiments, the biometric lock 20 is a fingerprint lock. For example, the lock 20 may have a fingerprint reader 22. The fingerprint reader 22 can store fingerprint information for one or more authorized users. By presenting their fingerprint to the fingerprint reader 22, an authorized user is able to unlock the lock 20 and access the internal volume 16 of the container. Other biometrics are known and can be used in place of or in addition to fingerprint (e.g., hand shape, facial recognition, iris, etc.) The use of biometric will eliminate the ability of users to share an unlock key or a code. The lock 20 may be configured to store an access log. In this way, when an authorized user operates the lock, a log entry is made with the user's identification (e.g., name, identification number, etc.) The access log may also include a time stamp for each entry showing the date and time the user operated the lock. This allows an audit trail for investigative or other purposes. In some embodiments, once the container has been unlocked and opened, the lock may be configured to re-lock when the container is closed (i.e., when the cover is secured to the housing). In some embodiments, once unlocked, the lock may be configured to re-lock after a predetermined period of time. In this way, even if the container is not opened, it will be re-locked after a predetermined period of time (e.g., 5 seconds, 10 seconds, or other times shorter, longer, or in between these times).

The container 10 includes a fastener 30 for securing the housing 12 to an IV pole 95. The fastener may secure the housing directly to an IV pole or indirectly (e.g., the cover may be secured to an IV pole and the housing indirectly secured via the cover). The fastener 30 may be a clamp, a bolt, a screw, a rivet, a strap, a buckle, a latch, or any other type of fastener suitable for such purposes. In some embodiments, the fastener is removable such that the housing can be removed from the IV pole. For example, upon access to the internal volume of the container, an authorized user may remove the fastener in order to remove the container from an IV pole. In some embodiments, the fastener is permanent. For example, the fastener may be a weld, adhesive, epoxy, etc. In some embodiments with a permanent fastener, the container may include an IV pole. The container may include more than one fastener. In some embodiments, the container is configured such that the IV pole is disposed through the container (for example, as depicted in FIG. 1. In other embodiments, the container is attached such that the IV pole is external to the container volume. The container may be configured to be attached anywhere along a length of the IV pole—for example, a middle portion of the IV pole, a top portion of the IV pole, or anywhere therebetween.

The container 10 may include a processor 40. For example, the processor may make up a portion of the lock or may be separate from the lock. The container 10 may include a communication port 42. The communication port 42 may be in electronic communication with the processor 40. The communication port may be wired or wireless. For example, the communication port may be a Bluetooth transceiver, a Wi-Fi transceiver, or other wireless port. The processor may be configured (e.g., programmed) to communication with a medication management system by way of the communication port. In this way, information can be exchanged with the medication management system such as, for example, access log information, medication used, medication location, etc. For example, the medication management system may be a Pyxis system (BD) or similar system.

In some embodiments, the lock operates as a self-contained unit. In other words, the lock is able to authenticate users and/or log access without communication with another device. In some embodiments, the lock communicates with another device/system (e.g., medication management system, etc.) in order to authenticate and/or log access. In some embodiments, the lock may be able to operate in either self-contained or connected mode. For example, the lock may normally operate in connected mode (communicating with a medication management system) but will operate in a self-contained mode if communication is lost for any reason. In some embodiments where the lock operates in either mode, once communications is re-established, the lock may transmit stored log information for the period when communication was disconnected.

The container 10 may further include a battery 44 to electrically power the lock. The battery may be the primary source of power or may be a backup power source for times when disconnected from line power. The battery may be a rechargeable battery. The container may operate similar to the line-and battery-powered infusion pumps commonly used today. The term battery should be broadly interpreted to include batteries, supercapacitors, and other forms of energy storage devices.

The container 10 may further include an alarm 50. The alarm may be an audible alarm (e.g., emitting a sound when triggered), a visual alarm (e.g., a light, flashing light, etc.), an signal sent to another device, or other type of alarm or combinations of alarms. The alarm may be configured to be triggered when the cover has been removed from the housing (i.e., the container is open) for a time greater than a predetermined period of time. For example, the alarm may be triggered if the cover has been removed for more than 60 seconds. In some embodiments, the alarm may change (become louder, change in tone, become brighter, etc.) if the cover has been removed for a time greater than a second predetermined period of time. For example, the alarm may be triggered if the container has remained open for more than 60 seconds, and the alarm may become louder if the cover has remained open for an additional 60 seconds (total of 120 seconds). Other predetermined time thresholds may be used. The predetermined time(s) may be programmable so that the alarm tailored for specific applications (e.g., a hospital unit that requires more complex IV configurations may program a longer predetermined time before the alarm sounds).

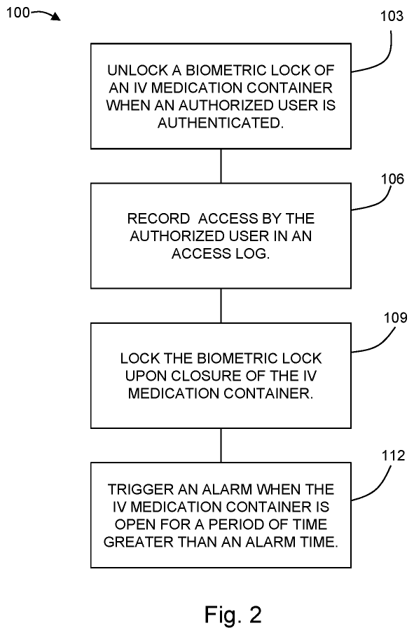

In another aspect (FIG. 2), the present disclosure may be embodied as a method 100 for securing IV medication during use. The method may include any of the steps described above. For example, the method 100 may include unlocking 103 a biometric lock of an IV medication container when an authorized user is authenticated (e.g., upon presentation of a fingerprint and/or other biometric). The IV medication container is secured to an IV pole. In other words, the IV medication container is configured for holding medication during use (e.g., during infusion of the medicament to a patient). The method 100 includes recording 106 access by the authorized user in an access log. The access log may be maintained (e.g., located) in the IV medication container, in a centralized medication management system (e.g., Pyxis), and/or otherwise. The biometric lock is locked 109 upon closure of the IV medication container. In some embodiments, the method 100 includes triggering 112 an alarm when a period of time between unlocking the biometric lock and closure of the IV medication container exceeds a predetermined alarm time.

Although the present disclosure has been described with respect to one or more particular embodiments, it will be understood that other embodiments of the present disclosure may be made without departing from the spirit and scope of the present disclosure.

1. A container for securing IV medication during use, comprising:
a housing having a removable cover, the housing having an internal volume for placement of IV medication;
a biometric lock for selectively locking the cover to the housing;
a fastener for affixing the housing to an IV pole.

2. The container of claim 1, wherein the removable cover is hingedly attached to the housing.

3. The container of claim 1, wherein the biometric lock is a fingerprint lock.

4. The container of claim 1, wherein the fastener is configured to be removable and is only operable from within the housing.

5. The container of claim 4, wherein the fastener is one or more of a clamp, a screw, a bolt, a latch, a buckle, and a strap.

6. The container of claim 1, further comprising an IV pole and wherein the fastener permanently attaches the housing to the IV pole.

7. The container of claim 1, wherein the biometric lock is configured to store an access log.

8. The container of claim 1, further comprising a processor.

9. The container of claim 8, further comprising a communication port in electronic communication with the processor.

10. The container of claim 9, wherein the processor is configured to communicate with a medication management system by way of the communication port.

11. The container of claim 1, further comprising a hangar for holding an IV bag.

12. The container of claim 1, wherein at least a portion of the cover is transparent.

13. The container of claim 12, wherein at least a portion of the cover is tinted.

14. The container of claim 1, further comprising an alarm.

15. The container of claim 14, wherein the alarm is configured to be triggered when the cover has been removed from the housing for a time greater than a predetermined period of time.

16. The container of claim 1, further comprising a battery for powering the biometric lock.

17. The container of claim 16, wherein the battery is rechargeable.

18. A method for securing IV medication during use, the method comprising:
unlocking a biometric lock of an IV medication container when an authorized user is authenticated, wherein the IV medication container is secured to an IV pole;
recording access by the authorized user in an access log; and
locking the biometric lock upon closure of the IV medication container.

19. The method of claim 18, further comprising triggering an alarm when a period of time between unlocking the biometric lock and closure of the IV medication container exceeds a predetermined alarm time.

20. The method of claim 18, wherein the access log is maintained by a medication management system in communication with the biometric lock.
